(19) United States
(12) Patent Application Publication
(10) Pub. No.: US 2017/0209385 A1
(43) Pub. Date: Jul. 27, 2017

(54) ETHYLCELLULOSE DISPERSION AND FILM

(71) Applicant: Dow Global Technologies LLC, Midland, MI (US)

(72) Inventors: True L. Rogers, Midland, MI (US); Stacey L. Dean, Litchfield, OH (US); Jason J. Folkenroth, Midland, MI (US); David L. Malotky, Midland, MI (US); Shari L. Workentine, Midland, MI (US)

(73) Assignee: Dow Global Technolgies LLC, Midland, MI (US)

(21) Appl. No.: 15/326,608

(22) PCT Filed: Jul. 2, 2015
(86) PCT No.: PCT/US15/38951

§ 371 Date: Jan. 16, 2017

Related U.S. Application Data
(60) Provisional application No. 62/025,739, filed on Jul. 17, 2014.

(51) Int. Cl. A61K 9/50; A61K 47/10; A61K 47/14; A61K 9/10; A61K 47/38

(52) U.S. Cl. CPC A61K 9/5047; A61K 9/10; A61K 47/38; A61K 47/14; A61K 47/10; A61K 9/5015

(57) ABSTRACT

Provided is an aqueous composition comprising (a) dispersed particles that comprise one or more ethylcellulose polymers and (b) one or more additives having molecular weight less than 220 g/mol, Hansen hydrogen bonding parameter greater than 11 MPa^{1/2} and less than 17.9 MPa^{1/2}, Hansen total solubility parameter greater than 22 MPa^{1/2}, and solubility in water greater than 2 g/L at 25° C. Also provided is a composition comprising particles having a coating, wherein said coating comprises 0-5% water by weight based on the weight of said coating, wherein said coating additionally comprises the ingredients (a) and (b).

It is often desirable to make films that contain ethylcellulose polymer. Such films are useful, for example, as coatings applied to other films or to multiparticulates. In some cases, a collection of beads contains a drug, and each of those beads is then coated with a film that contains ethylcellulose polymer. The film that contains ethylcellulose polymer can provide modified release of the drug when the beads are placed in an aqueous environment such as that of the gastrointestinal tract. It is also desirable that the film have good mechanical properties such as high tensile strength, high tensile elongation, and surface smoothness. The good mechanical properties allow the film to form a strong, flexible coating that provides a barrier around each bead that withstands common mechanical stresses.

In the past, a common method of making a film with good mechanical properties was to contact the beads with a solution in which ethylcellulose polymer was dissolved in an organic solvent. Organic solvents are undesirable because of adverse environmental and health effects. It is desired to provide an aqueous composition that contains ethylcellulose polymer and that is capable of producing high quality films. Additionally, in the past it was common to mix the ethylcellulose polymer with one or more plasticizers. Typical plasticizers were non-polymeric organic esters of molecular weight higher than 220 g/mol. It is desired to provide an aqueous composition that contains ethylcellulose polymer and that is capable of producing high quality films without the need for such plasticizers.

Tarvainen, et al. (“Enhanced film-forming properties for ethyl cellulose and starch acetate using n-alkyl succinic anhydrides as novel plasticizers,” European Journal of Pharmaceutical Sciences, vol. 19 (2000), pages 363-371) teach compositions in which Aquacoat™ dispersion (FMC Corporation) (an ethylcellulose polymer aqueous dispersion) is mixed with several plasticizers, all of which are relatively high molecular weight. It is desired to provide compositions using compounds of molecular weight lower than 220 g/mol.

The following is a statement of the invention.

The first aspect of the present invention is an aqueous composition comprising (a) dispersed particles that comprise one or more ethylcellulose polymers and (b) one or more additives having molecular weight less than 220 g/mol, Hansen hydrogen bonding parameter greater than 11 MPa^{1/2} and less than 17.9 MPa^{1/2}, Hansen total solubility parameter greater than 22 MPa^{1/2}, and solubility in water greater than 2 g/L at 25° C.

The second aspect of the present invention is a composition comprising particles having a coating, wherein said coating comprises 0-5% water by weight based on the weight of said coating, and wherein said coating additionally comprises (a) one or more ethylcellulose polymers and (b) one or more additives having molecular weight less than 220 g/mol, Hansen hydrogen bonding parameter greater than 11 MPa^{1/2} and less than 17.9 MPa^{1/2}, Hansen total solubility parameter greater than 22 MPa^{1/2}, and solubility in water greater than 2 g/L at 25° C.

The following is a detailed description of the invention.

As used herein, the following terms have the designated definitions, unless the context clearly indicates otherwise.

The Hansen total solubility parameter is a well known characteristic of molecules. It is defined, for example, in Hansen solubility parameters: A User's Handbook, by Charles M. Hansen, CRC Press, 1999. The Hansen total solubility parameter is normally represented by the lower case greek letter delta (δ), and it is reported in units of MPa^{1/2}. The Hansen total solubility parameter is derived from three other parameters as follows:

δ=\sqrt{δₕ²+δₚ²+δ_{d}²}

where δₕ is the hydrogen bonding parameter, δₚ is the dipole parameter, and δ_{d} is the dispersion parameter. The parameters δₕ, δₚ, and δ_{d}, are also reported in units of MPa^{1/2}.

For many compounds, the values of the Hansen total solubility parameter and the hydrogen bonding parameter may be obtained from one or more published tables of Hansen Solubility Parameters, including Hansen Solubility Parameters: A User's Handbook, by Charles M. Hansen, CRC Press, 1999; CRC Handbook of Solubility Parameters and Other Cohesion Parameters, second edition, by Allan F. M. Barton, CRC Press, 1991; and Polymer Handbook, by J. Brandrup, Wiley Interscience, 2003. If different values of the Hansen total solubility parameter or Hansen hydrogen bonding parameter appear in more than one published reference, the most recent reference is considered herein to be definitive.

Compounds are also characterized by the parameter log P, which is the base-10 logarithm of the ratio of the octanol-water partition coefficient. Values of log P for various compounds are tabulated in P
http://nanobionano.unibo.it/ChOrgInquinanti/media/OctanolWaterPartition_16_04_86.pdf and
http://www.crcnetbase.com/doi/abs/10.1201/b10501-92.
If different values of the log P parameter appear in more than one published reference, the most recent reference is considered herein to be definitive.

As used herein, an aqueous composition has 30% or more water by weight based on the weight of the composition. As used herein, a dispersion is a composition that contains a continuous medium that is liquid at 25° C. and contains discrete particles (herein called the “dispersed particles”) of a substance that are distributed throughout the continuous liquid medium. As used herein, an aqueous dispersion is an aqueous composition that is a dispersion in which the continuous liquid medium contains 50% or more water by weight based on the weight of the continuous liquid medium. Substances that are dissolved in the continuous liquid medium are considered herein to be part of the continuous liquid medium. The collection of all the dispersed particles is known herein as the “solid phase” of the dispersion. As used herein, the terms “dispersion” and “emulsion” are synonymous.

As used herein, a dispersant is a composition that improves the ability of the dispersed particles in a dispersion to become dispersed (that is, distributed throughout the continuous liquid medium) and/or to remain dispersed upon exposure to storage at 25° C., storage at temperatures of 25° C. to 45° C., exposure to shear, or a combination thereof.

As used herein, the “solids content” of an aqueous composition is the amount of material that remains when water and compounds having boiling point of 150° C. or less have been removed, by weight based on the total weight of the aqueous composition.

As used herein, the term high internal phase emulsion refers to an emulsion having equal to or greater than 74 wt % dispersed phase based on the total weight of the emulsion.

Ethylcellulose polymer, as used herein, means a derivative of cellulose in which some of the hydroxyl groups on the repeating glucose units are converted into ethyl ether groups. The number of ethyl ether groups can vary. The USP monograph requirement for ethyl ether content is from 44 to 51%.

As used herein, the viscosity of an ethylcellulose polymer is the viscosity of a 5 weight percent solution of that ethylcellulose polymer in a solvent, based on the weight of the solution. The solvent is a mixture of 80% toluene and 20% ethanol by weight based on the weight of the solvent. The viscosity of the solution is measured at 25° C. in an Ubbelohde viscometer.

As used herein, a fatty acid is a compound having a carboxyl group and a fatty group. A fatty group is a linear or branched chain of carbon atoms connected to each other that contains 8 or more carbon atoms. A hydrocarbon fatty group contains only carbon and hydrogen atoms.

As used herein, the “solubility in water” of a compound is the maximum amount of that compound that can be dissolved in water at 25° C., reported as grams of compound per liter of solution.

As used herein, an “ionic group” is a chemical group attached to a compound. When a compound that contains an ionic group is placed in water at 25° C., there exists some range of pH values (the “ionic pH range”) over which 50 mole percent or more of the ionic groups exists in an ionic state, and some or all of the ionic pH range falls within the pH range of 3 to 11.

As used herein, a “multiparticulate” is a plurality of particles. Particles are solid at 25° C. Particles are spherical or nearly spherical. If a particle is not spherical, its diameter is taken herein to be the diameter of a sphere having the same volume.

Any ethylcellulose polymer may be used in the present invention. The ethyl ether content of the ethylcellulose polymer is 44% or more; preferably 46% or more; more preferably 48% or more. The ethyl ether content of the ethylcellulose polymer is 51% or less; preferably 50% or less.

The ethylcellulose polymer preferably has viscosity of 2 mPa-s or higher; more preferably 5 mPa-s or higher; more preferably 10 mPa-s or higher; more preferably 15 mPa-s or higher. The ethylcellulose polymer preferably has viscosity of 120 mPa-s or lower; more preferably 100 mPa-s or lower; more preferably 80 mPa-s or lower; more preferably 60 mPa-s or lower; more preferably 40 mPa-s or lower; more preferably 30 mPa-s or lower.

Commercially available forms of ethylcellulose polymer which may be used in the invention include, for example, those available under the name ETHOCEL™, from The Dow Chemical Company. The ethylcellulose polymers used in the inventive examples are commercially available from The Dow Chemical Company as ETHOCEL™ Standard 4, ETHOCEL™ Standard 10, ETHOCEL™ Standard 20, ETHOCEL™ Standard 45, or ETHOCEL™ Standard 100 with ethyl ether content of from 48.0 to 49.5%. Other commercially available ethylcellulose polymers useful in embodiments of the invention include certain grades of AQUALON™ ETHYLCELLULOSE, available from Ashland, Inc., and certain grades of ASHACEL™ ethylcellulose polymers, available from Asha Cellulose Pvt.Ltd.

Optionally, any non-water-soluble cellulose derivative polymer may be used in addition to the ethylcellulose polymer.

The present invention involves an aqueous dispersion. Preferably, the continuous liquid medium contains water in the amount, by weight based on the weight of the continuous liquid medium, of 60% or more; more preferably 70% or more; more preferably 80% or more; more preferably 90% or more.

Preferably, the dispersed particles in the aqueous dispersion contain ethylcellulose polymer in an amount, by weight based on the total dry weight of the solid phase, of 45% or more; more preferably 50% or more; more preferably 55% or more; more preferably 60% or more; more preferably 65% or more. Preferably, the dispersed particles in the aqueous dispersion contain ethylcellulose polymer in an amount, by weight based on the total dry weight of the solid phase, of 95% or less; more preferably 90% or less; more preferably 85% or less; more preferably 80% or less.

Preferably, the composition of the present invention contains one or more dispersants. Preferred dispersants are fatty acids, which may be saturated or unsaturated. More preferred are unsaturated fatty acids. The fatty group of the fatty acid may be linear or branched; preferred is linear. The fatty group of the fatty acid may be a hydrocarbon fatty group or may have one or more substituent other than hydrogen or carbon; preferred are hydrocarbon fatty groups. Among unsaturated fatty acids, preferred are myristoleic acid, palmitoleic acid, sapienic acid, oleic acid, linoleic acid, and arachidonic acid. Among saturated fatty acids, preferred are caprylic acid, capric acid, lauric acid, palmitic acid, myristic acid, stearic acid, and arachidic acid. Preferably the dispersant contains oleic acid.

When a dispersant is present, the amount of dispersant is preferably, by weight based on the total dry weight of the solid phase, 2% or more; more preferably 7% or more; more preferably 8% or more. When a dispersant is present, the amount of dispersant is preferably, by weight based on the total dry weight of the solid phase, 20% or less; more preferably 18% or less.

The composition of the present invention contains one or more additives (b). Additive (b) is a compound that meets all of the following criteria: molecular weight less than 220 g/mol; Hansen hydrogen bonding parameter greater than 11 MPa^{1/2} and less than 17.9 MPa^{1/2}; Hansen total solubility parameter greater than 22 MPa^{1/2}; and solubility in water of greater than 2 g/L at 25° C. Preferably, additive (b) has molecular weight greater than 50 g/mol; more preferably greater than 80 g/mol; more preferably greater than 100 g/mol. Preferably, additive (b) has Hansen total solubility parameter of less than 40 MPa^{1/2}; more preferably less than 24.5 MPa^{1/2}. Preferably, additive (b) has solubility in water less than 150 g/L; more preferably less than 90 g/L; more preferably less than 55 g/L; more preferably less than 50 g/L.

Preferably, additive (b) has log P value of less than 2; more preferably, less than 1.8. Preferably, additive (b) has log P value of 0 or greater; more preferably 0.5 or greater; more preferably 0.8 or greater; more preferably 1.0 or greater.

Preferably, additive (b) contains one or more aromatic rings. Preferably, additive (b) contains no carboxyl groups; more preferably, additive (b) contains no ionic groups. Preferably, additive (b) contains a pendant —OH group that is attached either to an aliphatic carbon atom or to an aromatic carbon atom. Preferably, additive (b) is an alcohol or an ester or a mixture thereof; more preferably additive (b) contains an alcohol and an ester.

The amount of additive (b), by weight based on the total dry weight of the solid phase, is preferably 5% or more; more preferably 10% or more; more preferably 15% or more; more preferably 20% or more. The amount of additive (b), by weight based on the total dry weight of the solid phase, is preferably 50% or less; more preferably 40% or less; more preferably 35% or less.

Optionally, the composition of the present invention contains one or more plasticizers selected from the group consisting of triglycerides, organic esters having molecular weight of greater than 220, and alkyl carboxylic acids. The amount of such a plasticizer may be 0% to 35% by weight based on the total dry weight of the solid phase. Preferably, the amount of such plasticizer, by weight based on the total dry weight of the solid phase, is either zero or is less than 5%; more preferably, is either zero or is less than 1%; more preferably, is either zero or is less than 0.1%.

The preferred process of making the aqueous composition of the present invention is as follows. The process comprises feeding ethylcellulose polymer and a dispersant into a melt and mix zone of an extruder wherein the ethylcellulose polymer and dispersant are melted and mixed together to form a melt; conveying the melt to an emulsification zone of the extruder in which the temperature and pressure are controlled; feeding a base and water into the emulsification zone wherein the melt is dispersed to form a high internal phase emulsion; conveying the emulsion to a dilution and cooling zone of the extruder; and feeding water into the dilution and cooling zone to dilute the high internal phase emulsion thereby forming an aqueous dispersion. The general process conditions and equipment which may be used to perform the process are disclosed in U.S. Pat. Nos. 5,539,021 and 5,756,659.

The aqueous composition of the present invention preferably has pH of 12 or lower; more preferably 11 or lower; more preferably 10 or lower. The aqueous composition of the present invention preferably has pH or 7 or higher; more preferably 8 or higher.

The dispersed particles in the aqueous composition of the present invention preferably have volume-average particle diameter of 1.1 micrometer or less; more preferably 1.0 micrometer or less; more preferably 0.9 micrometer or less; more preferably 0.8 micrometers or less. The dispersed particles in the aqueous composition of the present invention preferably have volume-average particle diameter of 80 nm or greater; more preferably 90 nm or greater. Particle diameter is measured by laser diffraction. A suitable instrument is the Coulter™ LS-230 particle size analyzer (Beckman Coulter Corp.).

The aqueous composition of the present invention preferably has a solids content, by weight based on the weight of the aqueous composition, of 5% or more; more preferably 10% or more; more preferably 15% or more; more preferably 20% or more. The aqueous composition of the present invention preferably has a solids content, by weight based on the weight of the aqueous composition, of 55% or less; more preferably 50% or less; more preferably 45% or less; more preferably 40% or less; more preferably 35% or less.

The viscosity of the aqueous composition of the present invention is measured at 25° C. using a Brookfield RV-II viscometer using an RV2 spindle at 50 rpm. Preferably the viscosity of the aqueous composition is 250 mPa-s or lower; 100 mPa-s or lower; more preferably 80 mPa-s or lower; more preferably 60 mPa-s or lower; more preferably 40 mPa-s or lower; more preferably 20 mPa-s or lower. Preferably the viscosity of the aqueous composition is 1 mPa-s or higher.

A preferred use for the aqueous composition of the present invention is to produce a film. The aqueous composition of the present invention is optionally mixed with additional ingredients; a layer of the aqueous composition of the present invention is applied to a surface, and the water is removed. The resulting film preferably contains residual water in an amount, by weight based on the weight of the film, of 0 to 5%; more preferably 0 to 2%; more preferably 0 to 1%; more preferably 0 to 0.5%.

The resulting film may be used for any purpose. A preferred purpose is as a pharmaceutical coating or a food coating; more preferred is a pharmaceutical coating; more preferred is a modified-release pharmaceutical coating. A preferred method of making a modified-release pharmaceutical coating is to provide a multiparticulate that contains a drug and apply a coating of the film on the particles. Preferred multiparticulates are sugar or microcrystalline cellulose that has a drug applied as a layer to the surface or sprayed onto the surface. Alternatively, multiparticulates may contain a drug located in the interior of the particles, for example if the multiparticulates are made by extrusion followed by spheronization of a mixture of the drug with the material that will be made into the multiparticulates. The coating formed by the film made from the aqueous composition of the present invention preferably forms a complete layer of coating on 50% or more of the particles (by number); more preferably, the coating forms a complete layer of coating on 75% or more of the particles (by number) Preferably, on 90% or more of the particles (by number), the coating covers 75% or more of the area of the surface of each particle.

The following are examples of the present invention.

An aqueous dispersion of ethylcellulose polymer was formed as follows.

The ethylcellulose polymer was ETHOCEL™ Std 20 ethylcellulose, which has viscosity of 18-22 mPa-s and ethyl ether content of 48 to 49.5%.

A Berstorff ZE25 twin screw extruder rotating at 450 rpm was used to combine a polymer phase with a water phase to create an ethylcellulose dispersion. The extruder has a 25 mm screw diameter and a length over diameter (L/D) ratio of 36.

The polymer phase contained ethylcellulose and oleic acid. The ethylcellulose polymer was delivered to the extruder feed throat by a Schenck Mechatron loss-in-weight feeder. The oleic acid was delivered to a mixing and conveying zone injector by an Isco syringe pump.

The polymer phase was then melted and conveyed down the extruder barrel to an emulsification zone where it was combined with an initial amount of water and a base to create a high internal phase emulsion. The base used for all inventive examples was 28% wt. ammonia (as NH₃). The emulsion was then conveyed down the extruder barrel to the diluting and cooling zone. The initial water, base, and dilution water were each supplied separately to the extruder with Isco syringe pumps.

The ratio of ethylcellulose polymer to oleic acid was 74 parts by weight of ethylcellulose polymer to 9 parts by weight of oleic acid.

The ethylcellulose dispersion had pH=9.02; solids content of 26.53% by weight based on the total weight of the dispersion; viscosity (measured by Brookfield RV-II viscometer with RV2 spindle at 50 rpm at 25° C.) of 25 mPa-s; and volume-average particle diameter of 0.183 micrometers (measured with a Coulter LS230 particle size analyzer).

To produce aqueous compositions (Examples 1-4 and 9) of the present invention, the ethylcellulose dispersion was combined with additive (b) to give an amount of additive (b) of 27% by weight based on the solids content of the dispersion prior to addition of additive (b). That is, each aqueous composition had 27 parts by weight of additive (b), 89.2 parts by weight of ethylcellulose polymer, and 10.8 parts by weight of oleic acid. The mixture was stirred with an overhead propeller for 45-60 minutes.

To produce comparative compositions that used plasticizer instead of additive (b) (comparative examples 5C and 6C), the ethylcellulose dispersion was combined with plasticizer to give an amount of plasticizer of 27% by weight based on the solids content of the dispersion. The mixture was agitated with a homogenizer for 10 minutes. If attempts to achieve a homogeneous mixture of the ethylcellulose dispersion and the plasticizer using an overhead propeller failed; the rotor-stator homogenizer was required.

Comparative Example 7C was produced by adding triethyl citrate to Aquacoat™ dispersion (FMC) to produce a dispersion with 10% total solids in which the amount of triethyl citrate was 27% by weight based on the solids content of the dispersion.

To produce a comparative composition that was not aqueous (comparative example 8C), Ethocel™ Standard 20 Premium ethylcellulose polymer, oleic acid, and dibutyl sebacate were dissolved in organic solvent. The organic solvent was 80 weight % toluene and 20 weight % ethanol, based on the total weight of the solvent. The weight ratios of solids were 75 parts ethylcellulose polymer; 8.75 parts oleic acid, and 22.6 parts dibutyl sebacate. Total solids content of the solution was 30% by weight.

Films were cast at thickness of 0.5 mm (20 mil) wet onto a precleaned glass plate using a BYK four sided draw down bar. Films were covered and transferred to an oven set to 60° C. to cure for 2 hr. Films were then taken to a controlled humidity room (55% relative humidity, 22° C.) for at least 12 hrs for the moisture content of the films to equilibrate.

Films were removed from the substrate prior to tensile measurements. Tensile measurements were taken on 10 or more sample strips cut from at least three different films. The thickness of each sample strip was determined by measuring along three points using a Mitutoya Digimatic Indicator and averaging. The Young's modulus was measured by fitting the points in the linear area of the stress/strain curve. The maximum stress (reported as Tensile Strength) and strain at break (reported as % Elongation) were manually determined by reading the values from the stress/strain curve.

Tensile measurements were taken using an Instron™ frame 4201 tensile tester using a 50N static load cell (11 lb) equipped with smooth rubber grips. Prior to analysis, the films were held in a controlled humidity room (22° C., 50% RH) and allowed to equilibrate for a minimum of 12 hrs. Immediately prior to analysis, the films were removed from the glass substrates using a straight blade to lift and peel the films away from the surface of the glass plate. The films were punched using a pneumatic press using the ASTM D638 type V (dog bone) die. Each type of film was analyzed using ten samples cut from at least three different films. The thickness was determined by measuring along three points of the center of the film strips using a Mitutoya Digimatic™ Indicator and taking the average thickness. The strips were pulled at 0.508 cm/min (0.2 in/min).

Atomic Force Microscopy (AFM) data was collected of films still adhered to the glass substrate for all data except the free standing film cast from organic solvent. Peak Force Tapping Atomic Force Microscopy (PFT-AFM) images were obtained on a Veeco Icon using a Nanoscope V controller (software v 8.10). A Mikromasch NSC11 Side A cantilever was used. Height Sensor, Peak Force error, DMTModulus, LogDMTModulus, Adhesion, Deformation, Dissipation, & InPhase images in trace were captured and collected. Scanning conditions were peak force engage setpoint of 0.15 V and a peak force setpoint of 0.15 V (sample 3) and 0.24 V (sample 4) with a scan angle of 0° and a scan rate of 0.997 Hz. Images were acquired at a resolution of 512 scans/image and a scan size of 10×10 μm.

Surface roughness measurements were calculated for all three height images for each sample using Image Metrology SPIP software v.5.1.11. All processing of images obtained by AFM was performed using second-order polynomial plane fitting with mean z height set to zero; processing was performed on the height images prior to performing roughness calculations. Roughness values reported for each sample are based on an average calculated from three images per sample.

### EXAMPLE 1

### Tensile Testing and Surface Roughness

Test results were as follows:

TABLE 1
Tensile properties

| Ex- | | Young's | | Tensile |
|---|---|---|---|---|
| am- | | Modulus | % | Strength |
| ple | additive | (MPa^{1/2}) | Elongation | (MPa^{1/2}) |
| 1 | Phenylethyl alcohol | 444.8 ± 42.6 | 15.9 ± 2.7 | 20.2 ± 2.1 |
| | (PEA) | | | |
| 2 | Benzyl alcohol (BA) | 495 ± 32 | 14.9 ± 4.1 | 22.2 ± 1.2 |
| 3 | Propyl gallate | 32.4 ± 7.2 | 90.3 ± 9.5 | 5.3 ± 0.5 |
| 4 | 2-Phenoxyethanol | 54.3 ± 30.1 | 42.2 ± 12.2 | 3.1 ± 1.3 |
| 5C | triethyl citrate (TEC) | 20.7 ± 2.7 | 46.9 ± 15.8 | 0.9 ± 0.1 |
| 6C | dibutyl sebacate (DBS) | 23.1 ± 4.3 | 39.0 ± 8.9 | 1.3 ± 0.2 |
| 7C | (Aquacoat ™) TEC | 142 ± 38 | 9 ± 1 | 6 ± 2 |
| 8C | (solvent) DBS | 71.0 ± 3.7 | 33.7 ± 1.5 | 3.7 ± 0.1 |
| 9 | 1:1 PEA:BA by weight | 185 ± 29 | 45.7 ± 5.2 | 14.0 ± 1.7 |

TABLE 2
Surface Roughness

| Example | additive | Average Surface Roughness (Sₐ, nm) |
|---|---|---|
| 1 | Phenylethyl alcohol | 8.0 ± 2.0 |
| | (PEA) | |
| 2 | Benzyl alcohol (BA) | 5.5 ± 0.5 |
| 3 | Propyl gallate | 2.1 ± 0.3 |
| 4 | 2-Phenoxyethanol | 8.0 ± 1.0 |
| 5C | triethyl citrate (TEC) | 6.4 ± 0.8^{‡} |
| 6C | dibutyl sebacate (DBS) | 8.4 ± 2.8 |
| 7C | (Aquacoat ™) TEC | 2842.4 ± 377.7 |
| 8C | (solvent) DBS | 0.8 ± 0.2 |
| 9 | 1:1 PEA:BA by weight | 3.5 ± 0.8 |

Comparative Examples 5C and 6C (dispersions of Ethocel™ ethylcellulose with traditional plasticizers TEC and DBS) had inadequate tensile strength, significantly worse than all of the inventive examples. Comparative Example 7C (Aquacoat™ dispersion plus TEC) had poor tensile elongation and unacceptably high surface roughness.

Example 9 (blend of PEA and BA) has an especially desirable balance of tensile properties: tensile strength higher than 8 MPa and elongation higher than 20%.

### EXAMPLE 2

### Screening

A variety of additives were tested by preparing films as described above for preparing Examples 1-4, 5C, 6C, and 9. Except for the samples described above, films were not tested for tensile properties but were observed visually and handled manually to determine if a coalesced film formed. In order to be considered “coalesced,” a film had to form an intact film on the substrate and also had to remain intact upon removal from the substrate by hand without cracking or shattering. Results were as follows. Abbreviations used were these:
MW=molecular weight (g/mol)
Film?=Did the sample form a coalesced film? (yes or no)
δₕ=Hansen hydrogen bonding parameter (MPa^{1/2})
δ=Hansen total solubility parameter (MPa^{1/2})
W.Sol.=solubility in water at 25° C. (g/L)
log P=logarithm of the water-octanol partition coefficient (unitless)
ND=unknown

| | benzethonium | benzyl | benzyl | denatonium | dibutyl | diethanol- |
|---|---|---|---|---|---|---|
| | chloride | alcohol | benzoate | benzoate | sebacate | amine |
| MW | 448.1 | 108.1 | 212.2 | 446.6 | 314.5 | 105.1 |
| Film? | no | yes | yes | no | yes | no |
| δₕ | 4.7 | 12.6 | 4.7 | 7.3 | 4.9 | 18 |
| δ | 18.4 | 23.9 | 21.3 | 21.4 | 17.5 | 25.2 |
| W. Sol. | 40 | 42.9 | 0 | ND | 0.04 | 105 |
| logP | 4.00 | 1.05 | 3.81 | −0.088 | 5.958 | −1.761 |

| | diethyl | dimethyl | | linoleic | | |
|---|---|---|---|---|---|---|
| | phthalate | phthalate | glycerol | acid | maltitol | mannitol |
| MW | 222.2 | 194.2 | 92.1 | 280.4 | 344.3 | 182.2 |
| Film? | yes | yes | no | yes | no | no |
| δₕ | 4.5 | 4.6 | 24.2 | 5.1 | 60.1 | 44.8 |
| δ | 21.1 | 21.9 | 32.0 | 17.2 | 64.9 | 49.7 |
| W. Sol. | 1.08 | 1 | 500 | 0.00014 | 2000 | 182 |
| logP | 2.714 | 1.695 | −1.85 | 7.017 | −4.679 | −3.62 |

| | oleic | oleyl | 2-phenoxy | phenylethyl | polyethylene | poylvinyl |
|---|---|---|---|---|---|---|
| | acid | alcohol | ethanol | alcohol | glycol | alcohol |
| MW | 282.5 | 268.5 | 138.2 | 122.2 | 400 | 10000 |
| Film? | yes | yes | yes | yes | no | no |
| δₕ | 5.3 | 8 | 13.2 | 11.2 | 3.6 | 0.8 |
| δ | 17.4 | 18.2 | 23.9 | 22.6 | 19.8 | 26.2 |
| W. Sol. | 0 | 0 | 30 | 20 | ND | ND |
| logP | 7.421 | 7.562 | 1.247 | 1.504 | −4.8 | ND |

| | propyl | propylene | | stearic | succinic | |
|---|---|---|---|---|---|---|
| | gallate | glycol | sorbitol | acid | acid | triacetin |
| MW | 212.2 | 76.1 | 182.2 | 284.5 | 118.1 | 218.2 |
| Film? | yes | yes | no | yes | no | yes |
| δₕ | 16 | 23.3 | 44.8 | 5.3 | 19.8 | 9.6 |
| δ | 22.7 | 30.0 | 49.7 | 17.5 | 27.6 | 19.8 |
| W. Sol. | 3.5 | ND | 182 | 0.003 | 58 | 1.16 |
| logP | 1.779 | −1.008 | −3.262 | 7.83 | −0.933 | 0.216 |

| | tributyl citrate | triethanolamine | triethyl citrate | triolein | xylitol |
|---|---|---|---|---|---|
| MW | 360.4 | 149.2 | 276.3 | 885.4 | 152.1 |
| Film? | yes | no | yes | no | no |
| δₕ | 10.3 | 20 | 13.4 | 3.3 | 36.9 |
| δ | 19.9 | 27.9 | 21.8 | 16.8 | 42.7 |
| W. Sol. | ND | 149 | 65 | 0 | 1500 |
| logP | 4.324 | −0.988 | 1.267 | 23.44 | −2.65 |

1. An aqueous composition comprising
(a) dispersed particles that comprise one or more ethylcellulose polymers and
(b) one or more additives having molecular weight less than 220 g/mol, Hansen hydrogen bonding parameter greater than 11 MPa^{1/2} and less than 17.9 MPa^{1/2}, Hansen total solubility parameter greater than 22 MPa^{1/2}, and solubility in water greater than 2 g/L at 25° C.

2. The aqueous composition of claim 1, wherein said composition further comprises a dispersant.

3. The aqueous composition of claim 1, wherein said additive has log P of less than 2.

4. The aqueous composition of claim 1, wherein said additive comprises one or more benzene rings.

5. The aqueous composition of claim 1, wherein said additive comprises benzyl alcohol, phenylethyl alcohol, propyl gallate, phenoxy ethanol, or a mixture thereof.

6. The aqueous composition of claim 1, wherein said additive comprises benzyl alcohol and propyl gallate.

7. A process for coating a plurality of particles, said process comprising applying a layer of the aqueous composition of claim 1 to each of said particles.

8. A composition comprising particles having a coating, wherein said coating comprises 0-5% water by weight based on the weight of said coating, and wherein said coating additionally comprises
(a) one or more ethylcellulose polymers and
(b) one or more additives having molecular weight less than 220 g/mol, Hansen hydrogen bonding parameter greater than 11 MPa^{1/2} and less than 17.9 MPa^{1/2}, Hansen total solubility parameter greater than 22 MPa^{1/2}, and solubility in water greater than 2 g/L at 25° C.

9. The composition of claim 8, wherein said additive comprises benzyl alcohol, phenylethyl alcohol, propyl gallate, phenoxy ethanol, or a mixture thereof.

10. The composition of claim 8, wherein said additive comprises benzyl alcohol and propyl gallate.
